SEQUENCE LISTING (17 sequences)

SEQ ID NO: 1 - RNA, 21 nt, synthetic construct
  source 1..21: mol_type = other RNA
gcctgtagcccatgttgtatt

SEQ ID NO: 2 - RNA, 21 nt, synthetic construct
  source 1..21: mol_type = other RNA
tacaacatgggctacaggctt

SEQ ID NO: 3 - RNA, 21 nt, synthetic construct
  source 1..21: mol_type = other RNA
  modified_base 1: mod_base = OTHER; note = Lipophilic chain a modified guanosine
  modified_base 2..3: mod_base = s2c
  modified_base 4: mod_base = s2u
  modified_base 6: mod_base = s2u
  modified_base 7: mod_base = OTHER; note = 2-thioadenosine
  modified_base 9..11: mod_base = s2c
  modified_base 12: mod_base = OTHER; note = 2-thioadenosine
  modified_base 13: mod_base = s2u
  modified_base 15..16: mod_base = s2u
  modified_base 18: mod_base = s2u
  modified_base 19: mod_base = OTHER; note = 2-thioadenosine
gcctgtagcccatgttgtatt

SEQ ID NO: 4 - RNA, 21 nt, synthetic construct
  source 1..21: mol_type = other RNA
  modified_base 1: mod_base = OTHER; note = Lipophilic chain a modified uridine
tacaacatgggctacaggctt

SEQ ID NO: 5 - RNA, 21 nt, synthetic construct
  source 1..21: mol_type = other RNA
  modified_base 1: mod_base = OTHER; note = Lipophilic chain a modified guanosine
  modified_base 19: mod_base = OTHER; note = Lipophilic chain a modified guanosine
  modified_base 2..3: mod_base = s2c
  modified_base 4: mod_base = s2u
  modified_base 6: mod_base = s2u
  modified_base 7: mod_base = OTHER; note = 2-thioadenosine
  modified_base 9..11: mod_base = s2c
  modified_base 12: mod_base = OTHER; note = 2-thioadenosine
  modified_base 13: mod_base = s2u
  modified_base 15..16: mod_base = s2u
  modified_base 18: mod_base = s2u
gcctgtagcccatgttgtatt

SEQ ID NO: 6 - RNA, 21 nt, synthetic construct
  source 1..21: mol_type = other RNA
  modified_base 1: mod_base = OTHER; note = Lipophilic chain a modified uridine
  modified_base 19: mod_base = OTHER; note = Lipophilic chain a modified cytidine
tacaacatgggctacaggctt

SEQ ID NO: 7 - RNA, 21 nt, synthetic construct
  source 1..21: mol_type = other RNA
  modified_base 1: mod_base = OTHER; note = Lipophilic chain a modified guanosine
  modified_base 2: mod_base = OTHER; note = Lipophilic chain a modified cytidine
  modified_base 2..3: mod_base = s2c
  modified_base 4: mod_base = s2u
  modified_base 6: mod_base = s2u
  modified_base 7: mod_base = OTHER; note = 2-thioadenosine
  modified_base 9..11: mod_base = s2c
  modified_base 12: mod_base = OTHER; note = 2-thioadenosine
  modified_base 13: mod_base = s2u
  modified_base 15..16: mod_base = s2u
  modified_base 18: mod_base = s2u
  modified_base 19: mod_base = OTHER; note = 2-thioadenosine
gcctgtagcccatgttgtatt

SEQ ID NO: 8 - RNA, 21 nt, synthetic construct
  source 1..21: mol_type = other RNA
  modified_base 1: mod_base = OTHER; note = Lipophilic chain a modified uridine
  modified_base 2: mod_base = OTHER; note = Lipophilic chain a modified adenosine
tacaacatgggctacaggctt

SEQ ID NO: 9 - RNA, 21 nt, synthetic construct
  source 1..21: mol_type = other RNA
  modified_base 1: mod_base = OTHER; note = Lipophilic chain a modified guanosine
  modified_base 2: mod_base = OTHER; note = Lipophilic chain a modified cytidine
  modified_base 18: mod_base = OTHER; note = Lipophilic chain a modified uridine
  modified_base 19: mod_base = OTHER; note = Lipophilic chain a modified adenosine
  modified_base 3: mod_base = s2c
  modified_base 4: mod_base = s2u
  modified_base 6: mod_base = s2u
  modified_base 7: mod_base = OTHER; note = 2-thioadenosine
  modified_base 9..11: mod_base = s2c
  modified_base 12: mod_base = OTHER; note = 2-thioadenosine
  modified_base 13: mod_base = s2u
  modified_base 15..16: mod_base = s2u
gcctgtagcccatgttgtatt

SEQ ID NO: 10 - RNA, 21 nt, synthetic construct
  source 1..21: mol_type = other RNA
  modified_base 1: mod_base = OTHER; note = Lipophilic chain a modified uridine
  modified_base 2: mod_base = OTHER; note = Lipophilic chain a modified adenosine
  modified_base 18: mod_base = OTHER; note = Lipophilic chain a modified guanosine
  modified_base 19: mod_base = OTHER; note = Lipophilic chain a modified cytidine
tacaacatgggctacaggctt

SEQ ID NO: 11 - RNA, 21 nt, synthetic construct
  source 1..21: mol_type = other RNA
  modified_base 1: mod_base = OTHER; note = Lipophilic chain a modified guanosine
  modified_base 2..3: mod_base = s2c
  modified_base 4: mod_base = s2u
  modified_base 5: mod_base = OTHER; note = Lipophilic chain a modified guanosine
  modified_base 6: mod_base = s2u
  modified_base 7: mod_base = OTHER; note = 2-thioadenosine
  modified_base 8: mod_base = OTHER; note = Lipophilic chain a modified guanosine
  modified_base 9..11: mod_base = s2c
  modified_base 12: mod_base = OTHER; note = 2-thioadenosine
  modified_base 13: mod_base = s2u
  modified_base 14: mod_base = OTHER; note = Lipophilic chain a modified guanosine
  modified_base 15..16: mod_base = s2u
  modified_base 17: mod_base = OTHER; note = Lipophilic chain a modified guanosine
  modified_base 18: mod_base = s2u
  modified_base 19: mod_base = OTHER; note = 2-thioadenosine
gcctgtagcccatgttgtatt

SEQ ID NO: 12 - RNA, 20 nt, synthetic construct
  source 1..20: mol_type = other RNA
acaacatgggctacaggctt

SEQ ID NO: 13 - RNA, 21 nt, synthetic construct
  source 1..21: mol_type = other RNA
  modified_base 9..11: mod_base = OTHER; note = Lipophilic chain a modified guanosine
  modified_base 17..18: mod_base = OTHER; note = Lipophilic chain a modified guanosine
tacaacatgggctacaggctt

SEQ ID NO: 14 - RNA, 21 nt, synthetic construct
  source 1..21: mol_type = other RNA
  modified_base 1: mod_base = OTHER; note = Lipophilic chain b modified guanosine
  modified_base 5: mod_base = OTHER; note = Lipophilic chain b modified guanosine
  modified_base 8: mod_base = OTHER; note = Lipophilic chain b modified guanosine
  modified_base 14: mod_base = OTHER; note = Lipophilic chain b modified guanosine
  modified_base 17: mod_base = OTHER; note = Lipophilic chain b modified guanosine
  modified_base 2..3: mod_base = s2c
  modified_base 4: mod_base = s2u
  modified_base 6: mod_base = s2u
  modified_base 9..11: mod_base = s2c
  modified_base 13: mod_base = s2u
  modified_base 15..16: mod_base = s2u
  modified_base 18: mod_base = s2u
  modified_base 7: mod_base = OTHER; note = 2-thioadenosine
  modified_base 12: mod_base = OTHER; note = 2-thioadenosine
  modified_base 19: mod_base = OTHER; note = 2-thioadenosine
gcctgtagcccatgttgtatt

SEQ ID NO: 15 - RNA, 21 nt, synthetic construct
  source 1..21: mol_type = other RNA
  modified_base 1: mod_base = OTHER; note = Lipophilic chain c modified guanosine
  modified_base 5: mod_base = OTHER; note = Lipophilic chain c modified guanosine
  modified_base 8: mod_base = OTHER; note = Lipophilic chain c modified guanosine
  modified_base 14: mod_base = OTHER; note = Lipophilic chain c modified guanosine
  modified_base 17: mod_base = OTHER; note = Lipophilic chain c modified guanosine
  modified_base 7: mod_base = OTHER; note = 2-thioadenosine
  modified_base 12: mod_base = OTHER; note = 2-thioadenosine
  modified_base 19: mod_base = OTHER; note = 2-thioadenosine
  modified_base 2..3: mod_base = s2c
  modified_base 4: mod_base = s2u
  modified_base 6: mod_base = s2u
  modified_base 9..11: mod_base = s2c
  modified_base 13: mod_base = s2u
  modified_base 15..16: mod_base = s2u
  modified_base 18: mod_base = s2u
gcctgtagcccatgttgtatt

SEQ ID NO: 16 - RNA, 21 nt, synthetic construct
  source 1..21: mol_type = other RNA
  modified_base 1: mod_base = OTHER; note = Lipophilic chain d modified guanosine
  modified_base 5: mod_base = OTHER; note = Lipophilic chain d modified guanosine
  modified_base 8: mod_base = OTHER; note = Lipophilic chain d modified guanosine
  modified_base 14: mod_base = OTHER; note = Lipophilic chain d modified guanosine
  modified_base 17: mod_base = OTHER; note = Lipophilic chain d modified guanosine
  modified_base 7: mod_base = OTHER; note = 2-thioadenosine
  modified_base 12: mod_base = OTHER; note = 2-thioadenosine
  modified_base 19: mod_base = OTHER; note = 2-thioadenosine
  modified_base 2..3: mod_base = s2c
  modified_base 4: mod_base = s2u
  modified_base 6: mod_base = s2u
  modified_base 9..11: mod_base = s2c
  modified_base 13: mod_base = s2u
  modified_base 15..16: mod_base = s2u
  modified_base 18: mod_base = s2u
gcctgtagcccatgttgtatt

SEQ ID NO: 17 - RNA, 21 nt, synthetic construct
  source 1..21: mol_type = other RNA
  modified_base 1: mod_base = OTHER; note = Lipophilic chain e modified guanosine
  modified_base 5: mod_base = OTHER; note = Lipophilic chain e modified guanosine
  modified_base 8: mod_base = OTHER; note = Lipophilic chain e modified guanosine
  modified_base 14: mod_base = OTHER; note = Lipophilic chain e modified guanosine
  modified_base 17: mod_base = OTHER; note = Lipophilic chain e modified guanosine
  modified_base 7: mod_base = OTHER; note = 2-thioadenosine
  modified_base 12: mod_base = OTHER; note = 2-thioadenosine
  modified_base 19: mod_base = OTHER; note = 2-thioadenosine
  modified_base 2..3: mod_base = s2c
  modified_base 4: mod_base = s2u
  modified_base 6: mod_base = s2u
  modified_base 9..11: mod_base = s2c
  modified_base 13: mod_base = s2u
  modified_base 15..16: mod_base = s2u
  modified_base 18: mod_base = s2u
gcctgtagcccatgttgtatt

(19) United States
(12) Patent Application Publication
(10) Pub. No.: US 2026/0049099 A1
(43) Pub. Date: Feb. 19, 2026

(54) DOUBLE-STRANDED SIRNA, PREPARATION METHOD THEREOF, PHARMACEUTICAL COMPOSITION AND APPLICATION THEREOF

(71) Applicant: Yimeichengjian (Shanghai) Biomedical Co., Ltd., Shanghai (CN)

(72) Inventors: Ping LI, Shanghai (CN); Songlin XU, Shanghai (CN); Xueming TANG, Shanghai (CN)

(21) Appl. No.: 19/374,145

(22) Filed: Oct. 30, 2025

Related U.S. Application Data
(63) Continuation of application No. PCT/CN2025/087230, filed on Apr. 3, 2025.

(30) Foreign Application Priority Data
Dec. 3, 2024 (CN) 2024117549715

(51) Int. Cl. C07H 21/02; A61K 9/00; A61K 31/7105; A61K 31/713; C07H 15/04

(52) U.S. Cl. CPC C07H 21/02; A61K 9/0019; A61K 31/7105; A61K 31/713; C07H 15/04

(57) ABSTRACT

The present disclosure provides a double-stranded siRNA with lipophilic modification. The modified siRNA molecules exhibit excellent in vivo distribution and delivery efficiency via intrathecal injection, intradermal injection, or other administration routes. The molecules effectively knock down target gene expression while maintaining good tolerability and safety. The present disclosure further provides a preparation method of siRNA with lipophilical modification, a pharmaceutical composition, and application of the same in the preparation of a medicament for treating a TNF-α-mediated disease or disorder.

### REFERENCE TO SEQUENCE LISTING

The instant application contains a Sequence Listing in XML format as a file named “PC250009A.xml”, created on 2025 Oct. 29, of 77324 byte in size, and which is hereby incorporated by reference in its entirety.

### TECHNICAL FIELD

The present disclosure belongs to the field of biomedicine technology, and in particular to a double-stranded siRNA, a preparation method thereof, a pharmaceutical composition and application thereof in the preparation of a medicament for treating a TNF-α-mediated disease or disorder.

### BACKGROUND

Most of the RNAi drugs currently available on the market adopt lipid nanoparticles modified with N-acetylgalactosamine to target liver tissues. siRNA molecules are poorly taken up by cells due to their inherent hydrophilicity and negative charge. To apply the RNAi technology to other tissues (such as a central nervous system, eyes, and lungs), it is necessary to develop new technologies to improve the delivery and distribution performance of siRNA in extrahepatic tissues.

To date, various categories of siRNA carrier, including polymers and polypeptides, have been reported. For example, U.S. Pat. No. 9,061,995 B2 discloses a peptide conjugate for delivering nucleic acids (such as siRNA), where the conjugate includes a cell-penetrating peptide moiety designed to enhance intracellular delivery efficiency of nucleic acid therapeutics. Although these peptides demonstrate promising results under laboratory conditions, there remain numerous challenges in translating them into clinical applications. In particular, cell-penetrating peptides generally suffer from poor in vivo stability and are susceptible to degradation by enzymes, resulting in reduced efficacy. Moreover, large-scale synthesis and purification are technically complex and costly. With technological advancement, polymeric materials are also being developed. Polyethyleneimine (PEI) is a cationic polymer that binds to negatively charged nucleic acids to form nanoscale complexes. These complexes are capable of entering cells and releasing nucleic acids through endocytosis. Poly (lactic-co-glycolic) acid (PLGA) is a biodegradable copolymer with excellent biocompatibility and controllable degradation rate. It can encapsulate small nucleic acid therapeutics and deliver them to target tissues or cells through passive or active targeting mechanisms. However, some polymers may cause immune responses or cytotoxicity, leading to adverse side effects.

More drugs for extrahepatic delivery are under development. For pulmonary targeting, Arrowhead Pharmaceuticals has developed ARO-MMP7 for the treatment of pulmonary fibrosis, which delivers nucleic acid therapeutics to the lungs via inhalation. For ocular targeting, Regenxbio has developed ABBV-RGX-314 for the treatment of wet age-related macular degeneration. It delivers nucleic acid drugs into the eye via subretinal injection. This method is expected to be applied to treat glaucoma and diabetic retinopathy in the future. For tumor targeting, Amgen has developed AMG-160 for the treatment of solid tumors. It designs a responsive delivery system that specifically delivers small nucleic acid therapeutics to tumor tissues by leveraging the characteristics of a tumor microenvironment (such as pH or redox state). For cardiac targeting, Cardior Pharmaceuticals has developed CDR132L for the treatment of heart failure. For renal targeting, nanoparticle-encapsulated small nucleic acid therapeutics can be intravenously administered for delivery to kidney tissues to treat nephrotic syndrome and chronic kidney disease. For example, Silence Therapeutics has developed SLN501 for the treatment of IgA nephropathy.

Delivery of nucleic acid therapeutics to the nervous system is highly challenging, as a blood-brain barrier hinders effective drug transport of many conventional delivery methods. Nevertheless, nucleic acid therapeutics have shown great potential in treating neurological disorders, and many companies are sparing great efforts to research and develop nucleic acid therapeutics. Voyager Therapeutics has developed VY-HTT01, which uses an adeno-associated virus (AAV) vector to deliver siRNA targeting the Huntington (HTT) gene, thereby reducing expression of the mutant HTT protein. Preliminary results demonstrate safety and potential efficacy. Biogen has developed Spinraza, which is an antisense oligonucleotide (ASO) that modifies the splicing of SMN2 gene by intrathecal injection to increase production of functional SMN protein. Spinraza has been approved by the FDA and is widely used in the treatment of spinal muscular atrophy (SMA). UniQure has developed AMT-130, which uses an AAV vector to deliver miRNA targeting the Huntington (HTT) gene, reducing a-synuclein expression. AMT-130 is currently under clinical trials to evaluate its safety and efficacy.

Effective delivery of small nucleic acid therapeutics is highly dependent on the delivery system. The druggability of many small nucleic acids depends heavily on the development of suitable delivery vectors, which significantly constrains their druggability. Accordingly, developing an effective carrier-free siRNA delivery technology for extrahepatic tissues is the current research and development focus of siRNA drugs.

### SUMMARY

Improving the lipophilicity of a drug is an effective strategy to enhance cellular uptake of siRNA therapeutics and to achieve extrahepatic targeting. To improve the carrier-free delivery and distribution performance of siRNA in extrahepatic tissues, the present disclosure enhances the lipophilicity of siRNA molecules through a refined structural design, thereby improving its distribution performance and delivery effect.

In one aspect, the present disclosure provides a nucleoside having a structure as represented by Formula (I), including R₁, R₃, and lipophilic R₂, where:

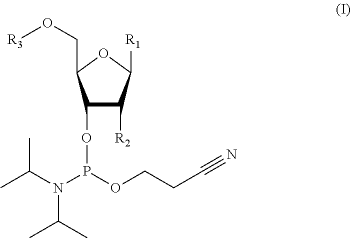

R₁ is selected from a nitrogenous base or a nitrogenous base analog.
R₂ is selected from the following groups:

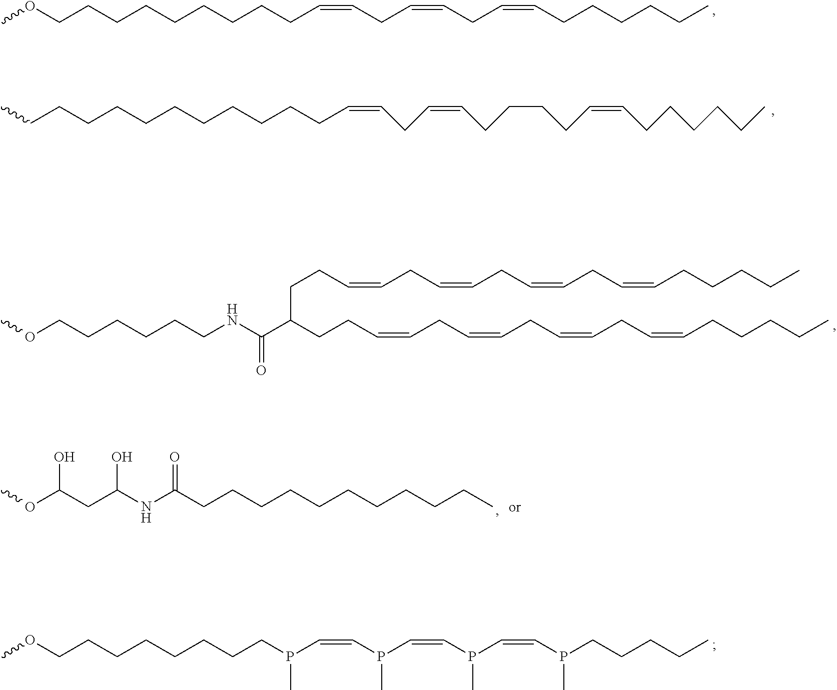

R₃ is selected from 4,4′-dimethoxytrityl (DMTr), 4-monomethoxytrityl (MMTr), 4,4′,4″-trimethoxytrityl (TMTr), pivaloyloxymethyl (PIVOM), tert-butyldimethylsilyl (TBDMS), 9-fluorenylmethoxycarbonyl (Fmoc), phenoxyacetyl (PAC), 4-tert-butylphenoxyacetyl (tBPAC), 2-cyanoethoxy-N,N-diisopropylaminophosphino (CEP), 3-pentanedione, acetyl, benzoyl, 2-cyanoethyl, N,N-dibutylaminocarbonyl (DBF), N,N-dimethylaminocarbonyl (DMF), isobutyryl, 2-(2-nitrophenyl)-propoxycarbonyl (NPPOC), triethylammonium (TEA), trifluoroacetyl, triisopropylsiloxymethyl (TOM), para-isopropylphenoxyacetyl (iPrPAC), O-acetal levulinyl ester (ALE), phenylacetyl, or 1,1′,3,3′-tetra-isopropyl disiloxanyl.

In another aspect, the present disclosure provides a double-stranded siRNA, including a sense strand and an antisense strand, where a sequence of the sense strand is 5′-GCCUGUAGCCCAUGUUGUATT-3′ whose nucleotide sequence is shown in SEQ ID NO.1, and a sequence of the antisense strand is 5′-UACAACAUGGGCUACAGGCTT-3′ whose nucleotide sequence is shown in SEQ ID NO.2; and a nucleotide in the double-stranded siRNA is specially modified. The specially modified nucleotide include R₁ and lipophilic R₂, having a structure as represented by Formula (II):

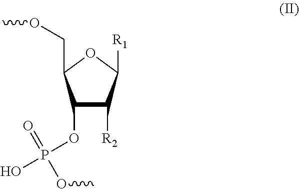

R₁ is selected from a nitrogenous base or a nitrogenous base analog.
R₂ is selected from the following groups:

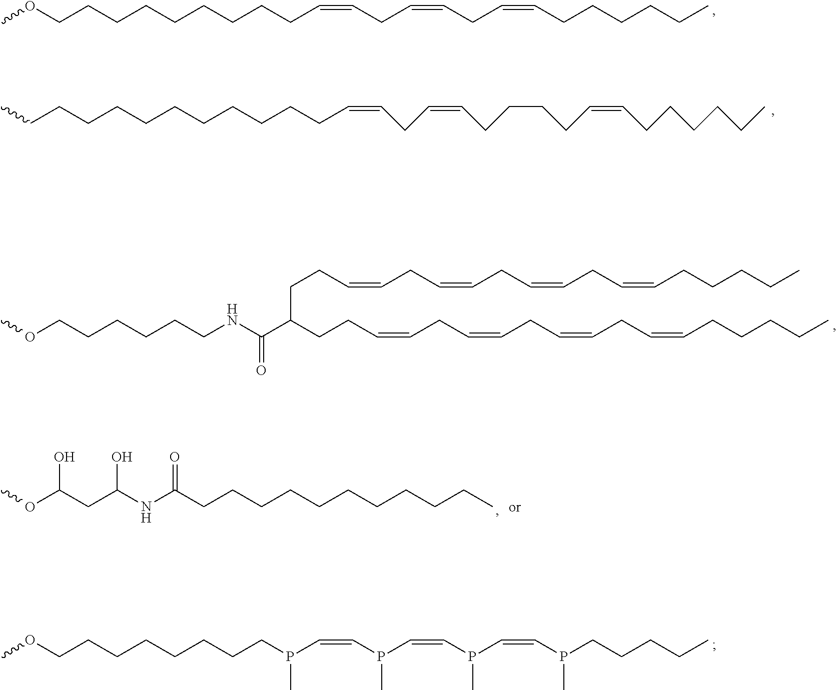

In one or more embodiments, the specially modified nucleotide is located at any one or more positions in a 5′ to 3′ direction of a sense strand whose nucleotide sequence is shown in SEQ ID NO.1 or an antisense strand whose nucleotide sequence is shown in SEQ ID NO.2.

In one or more embodiments, the specially modified nucleotide is located at one or more positions selected from positions 1, 2, 5, 8, 9, 10, 11, 14, 17, 18, or 19 in the 5′ to 3′ direction of the sense strand whose nucleotide sequence is shown in SEQ ID NO.1 or the antisense strand whose nucleotide sequence is shown in SEQ ID NO.2.

In one or more embodiments, the specially modified nucleotide is located at one or more positions selected from positions 1, 2, 5, 8, 14, 17, 18, or 19 in a 5′ to 3′ direction of the sense strand whose nucleotide sequence is shown in SEQ ID NO.1.

In one or more embodiments, the specially modified nucleotide is located at one or more positions selected from positions 1, 2, 9, 10, 11, 17, 18, or 19 in a 5′ to 3′ direction of the antisense strand whose nucleotide sequence is shown in SEQ ID NO.2.

In one or more embodiments, the specially modified nucleotide is located at:
position 1 in the 5′ to 3′ direction of the sense strand whose nucleotide sequence is shown in SEQ ID NO.1;
positions 1 and 19 in the 5′ to 3′ direction of the sense strand whose nucleotide sequence is shown in SEQ ID NO.1;
positions 1 and 2 in the 5′ to 3′ direction of the sense strand whose nucleotide sequence is shown in SEQ ID NO.1;
positions 1, 2, 18 and 19 in the 5′ to 3′ direction of the sense strand whose nucleotide sequence is shown in SEQ ID NO.1; or
positions 1, 5, 8, 14 and 17 in the 5′ to 3′ direction of the sense strand whose nucleotide sequence is shown in SEQ ID NO.1; or
In one or more embodiments, the specially modified nucleotide is located at:

position 1 in the 5′ to 3′ direction of the antisense strand whose nucleotide sequence is shown in SEQ ID NO.2;
positions 1 and 19 in the 5′ to 3′ direction of the antisense strand whose nucleotide sequence is shown in SEQ ID NO.2;
positions 1 and 2 in the 5′ to 3′ direction of the antisense strand whose nucleotide sequence is shown in SEQ ID NO.2;
positions 1, 2, 18 and 19 in the 5′ to 3′ direction of the antisense strand whose nucleotide sequence is shown in SEQ ID NO.2; or
positions 9, 10, 11, 17 and 18 in the 5′ to 3′ direction of the antisense strand whose nucleotide sequence is shown in SEQ ID NO.2.

In one or more embodiments, the specially modified nucleotide is located at:
position 1 in the 5′ to 3′ direction of the sense strand whose nucleotide sequence is shown in SEQ ID NO.1, and position 1 in the 5′ to 3′ direction of the antisense strand whose nucleotide sequence is shown in SEQ ID NO.2;
positions 1 and 19 in the 5′ to 3′ direction of the sense strand whose nucleotide sequence is shown in SEQ ID NO.1, and positions 1 and 19 in the 5′ to 3′ direction of the antisense strand whose nucleotide sequence is shown in SEQ ID NO.2;
positions 1 and 2 in the 5′ to 3′ direction of the sense strand whose nucleotide sequence is shown in SEQ ID NO.1, and positions 1 and 2 in the 5′ to 3′ direction of the antisense strand whose nucleotide sequence is shown in SEQ ID NO.2;
positions 1, 2, 18 and 19 in the 5′ to 3′ direction of the sense strand whose nucleotide sequence is shown in SEQ ID NO.1, and positions 1, 2, 18 and 19 in the 5′ to 3′ direction of the antisense strand whose nucleotide sequence is shown in SEQ ID NO.2;
positions 1, 5, 8, 14 and 17 in the 5′ to 3′ direction of the sense strand whose nucleotide sequence is shown in SEQ ID NO.1; or
positions 1, 5, 8, 14 and 17 in the 5′ to 3′ direction of the sense strand whose nucleotide sequence is shown in SEQ ID NO.1, and positions 9, 10, 11 17 and 18 in the 5′ to 3′ direction of the antisense strand whose nucleotide sequence is shown in SEQ ID NO.2.

In one or more embodiments,
a sequence of the sense strand is 5′-GCCUGUAGCCCAUGUUGUATT-3′ (SEQ ID NO.1), and a sequence of the antisense strand is 5′-UACAACAUGGGCUACAGGCTT-3′ (SEQ ID NO.2).

In one or more embodiments, a non-bridging oxygen atom of a phosphate group attached to a 3′-carbon atom in nucleoside monophosphate at one or more positions of the sense strand or the antisense strand is substituted with a sulfur atom.

In one or more embodiments, a non-bridging oxygen atom of a phosphate group attached to a 3′-carbon atom in nucleoside monophosphate at positions 2, 3, 4, 6, 7, 9, 10, 11, 12, 13, 15, 16, 18 and 19 of the sense strand whose nucleotide sequence is shown in SEQ ID NO.1 or the antisense strand whose nucleotide sequence is shown in SEQ ID NO.2 is substituted with a sulfur atom.

In yet another aspect, the present disclosure provides a method for preparing the nucleotide as described in any one of the embodiments herein, which includes: synthesizing a lipophilic chain and reacting the lipophilic chain with an dehydrated nucleotide to obtain the nucleotide as described in any one of the embodiments herein, where the dehydrated nucleotide is selected from dehydrated adenosine or an analog thereof, dehydrated uridine or an analog thereof, dehydrated cytosine or an analog thereof, dehydrated thymine or an analog thereof, dehydrated guanine or an analog thereof.

In still another aspect, the present disclosure provides a method for preparing a double-stranded siRNA as described in any one of the embodiments herein, including synthesizing RNA using the nucleotide as described in any one of the embodiments herein as a starting material.

In one or more embodiments, the method is a phosphite triester method.

In still another aspect, the present disclosure provides a pharmaceutical composition, which includes a therapeutically effective amount of the double-stranded siRNA as described in any one of the embodiments herein, a pharmaceutically acceptable carrier, a solvent or an excipient.

In still another aspect, the present disclosure provides a method for preparing a pharmaceutical composition as described in any one of the embodiments herein, and the method includes adding a double-stranded siRNA, a pharmaceutically acceptable carrier or an excipient to a solvent.

In one or more embodiments, the method includes adding the following components to a portion of the solvent based on the final volume: a concentration of 0.2-20 mg/ml of double-stranded siRNA, 0.02-1 mg/mL of calcium chloride dihydrate, 0.01-1 mg/mL of sodium dihydrogen phosphate, 0.01-1 mg/mL of magnesium chloride hexahydrate, 0.01-1 mg/ml of sodium phosphate, and 0.01-1 mg/mL of potassium chloride, dissolving thoroughly, and diluting the solution with the solvent to make up to the final volume, where the solvent is sterile water for injection, and finally adjusting a pH to 6.5-7.5 using 1 mg/mL of HCl or NaOH solution.

In one or more embodiments, the method includes adding the following components to a portion of the solvent based on the final volume: a concentration of 2 mg/mL of double-stranded siRNA, 0.2 mg/mL of calcium chloride dihydrate, 0.1 mg/mL of sodium dihydrogen phosphate, 0.16 mg/mL of magnesium chloride hexahydrate, 0.03 mg/mL of sodium phosphate, and 0.22 mg/mL of potassium chloride, dissolving thoroughly, and diluting the solution with the solvent to make up to the final volume, where the solvent is sterile water for injection, and finally adjusting a pH to 6.9-7.1 using 1 mg/mL of HCl or NaOH solution.

In still another aspect, the present disclosure provides application of the double-stranded siRNA as described in any one of the embodiments herein in the preparation of medicaments for treating a TNF-α-mediated disease or disorder. In one or more embodiments, the TNF-α-mediated disease or disorder is selected from inflammation, autoimmune diseases, hyperalgesia, and cancer. More preferably, the TNF-α-mediated disease or disorder is selected from psoriasis, psoriatic arthritis, spondylitis, myelitis, encephalitis, systemic lupus erythematosus, arthritis, inflammatory bowel disease, central nervous system hypersensitivity, peripheral nerve system hypersensitivity, leukemia, lymphoma, melanoma, gastric cancer, liver cancer, gallbladder cancer, renal cancer, lung cancer, myeloma, reproductive system cancer, breast cancer, pancreatic cancer, bone cancer, or head and neck tumors.

The present disclosure connects a lipophilic carbon chain to the 2′-C terminus of the pentose in the nucleotide monomer in the siRNA, thereby increasing the lipophilicity of the siRNA sequence, improving its carrier-free delivery effect and distribution performance, without negatively affecting its efficacy and safety. The siRNA modified with a lipophilic chain can more effectively knock down the expression of the target gene.

### BRIEF DESCRIPTION OF THE DRAWINGS

FIG. 1A is a ¹H-NMR spectrum of a lipophilic chain a.

FIG. 1B is a ¹³C-NMR spectrum of a lipophilic chain a.

FIG. 2A is a ¹H-NMR spectrum of a lipophilic chain b.

FIG. 2B is a ¹³C-NMR spectrum of a lipophilic chain b.

FIG. 3A is a ¹H-NMR spectrum of a lipophilic chain c.

FIG. 3B is a ¹³C-NMR spectrum of a lipophilic chain c.

FIG. 4A is a ¹H-NMR spectrum of a lipophilic chain d.

FIG. 4B is a ¹³C-NMR spectrum of a lipophilic chain d.

FIG. 5A is a ¹H-NMR spectrum of a lipophilic chain e.

FIG. 5B is a ¹³C-NMR spectrum of a lipophilic chain e.

FIG. 6 is an exemplary synthetic route of nucleoside A.

FIG. 7A is a structural diagram of nucleoside A.

FIG. 7B is a structural diagram of nucleoside B.

FIG. 7C is a structural diagram of nucleoside C.

FIG. 7D is a structural diagram of nucleoside D.

FIG. 7E is a structural diagram of nucleoside E.

FIG. 8A shows changes in target gene mRNA expression levels in brain cells of mice after transfection with 1 #-6 #.

FIG. 8B shows changes in target gene mRNA expression levels in brain cells of mice after transfection with 5 #B-5 #E.

FIG. 9A shows changes in target gene mRNA expression levels in various organs of mice after intrathecal injection of 5 #.

FIG. 9B shows changes in target gene mRNA expression levels in various organs of mice after intrathecal injection of 5 #A.

FIG. 9C shows changes in target gene mRNA expression levels in various organs of mice after intrathecal injection of 5 #B.

FIG. 9D shows changes in target gene mRNA expression levels in various organs of mice after intrathecal injection of 5 #C.

FIG. 9E shows changes in target gene mRNA expression levels in various organs of mice after intrathecal injection of 5 #D.

FIG. 9F shows changes in target gene mRNA expression levels in various organs of mice after intrathecal injection of 5 #E.

FIG. 10 shows changes in target gene mRNA expression levels in skin tissues of mice after intradermal injection of siRNA.

FIG. 11 shows changes in cell viability of HaCat cells after transfection with siRNA.

FIG. 12A shows fluorescence images of various mouse tissues after intrathecal injection of 5 #A.

FIG. 12B shows a corresponding color scale.

### DETAILED DESCRIPTIONS OF THE EMBODIMENTS

### Nucleosides

The present disclosure provides a nucleoside having a structure as represented by Formula (I), including R₁, R₃, and lipophilic R₂, where:

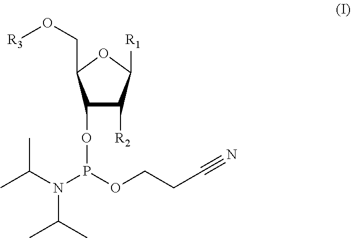

R₁ is selected from a nitrogenous base or a nitrogenous base analog.

Preferably, R₁ is selected from adenine, uracil, cytosine, or guanine. Preferably, R₁ is selected from adenine, uracil, cytosine, or guanine modified by one or more of thiolation, halogenation, hydroxylation, amination, carboxylation, or methylation. Preferably, R₁ is selected from adenine methyl, uracil methyl, cytosine methyl, or guanine methyl modified by one or more of thiolation, halogenation, hydroxylation, amination, carboxylation, or methylation. Preferably, R₁ is selected from adenine formyl, uracil formyl, cytosine formyl, or guanine formyl modified by one or more of thiolation, halogenation, hydroxylation, amination, carboxylation, or methylation.

R₂ is selected from H, C₁₂-C₅₀ alkyl, or C₁₂-C₅₀ alkyl modified by one or more of the following groups: hydroxyl, ether, carbonyl, amide, carboxyl, ester, amino, imino, tertiary amino, halogen, thio, thioether, sulfhydryl, sulphonic acid group, phospho, or phosphate groups.

Preferably, R₂ is selected from the following groups:

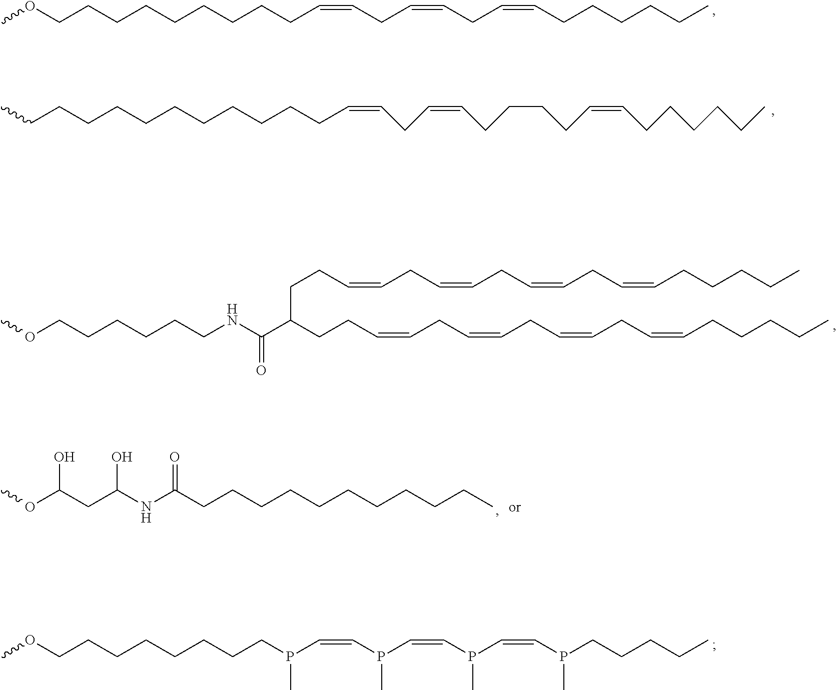

R₃ is selected from 4,4′-dimethoxytrityl (DMTr), 4-monomethoxytrityl (MMTr), 4,4′,4″-trimethoxytrityl (TMTr), pivaloyloxymethyl (PivOM), tert-butyldimethylsilyl (TBDMS), 9-fluorenylmethoxycarbonyl (Fmoc), phenoxyacetyl (PAC), 4-tert-butylphenoxyacetyl (tBPAC), 2-cyanoethoxy-N,N-diisopropylaminophosphino (CEP), 3-pentanedione, acetyl, benzoyl, 2-cyanoethyl, N,N-dibutylaminocarbonyl (DBF), N,N-dimethylaminocarbonyl (DMF), isobutyryl, 2-(2-nitrophenyl)-propoxycarbonyl (NPPOC), triethylammonium (TEA), trifluoroacetyl, triisopropylsiloxymethyl (TOM), para-isopropylphenoxyacetyl (iPrPAC), O-acetal levulinyl ester (ALE), phenylacetyl, or 1,1′,3,3′-tetra-isopropyl disiloxanyl. Preferably, R₃ is 4,4′-dimethoxytrityl (DMTr).
   Double-Stranded siRNA

The present disclosure provides a double-stranded siRNA, including a sense strand and an antisense strand, where a nucleotide in the double-stranded siRNA is specially modified. The specially modified nucleotide includes R₁ and lipophilic R₂, having a structure as represented by Formula (II):

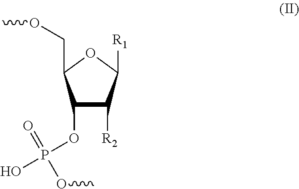

R₁ is selected from a nitrogenous base or a nitrogenous base analog. Preferably, R₁ is selected from adenine, uracil, cytosine, or guanine.

Preferably, R₁ is selected from adenine, uracil, cytosine, or guanine modified by one or more of thiolation, halogenation, hydroxylation, amination, carboxylation, or methylation. Preferably, R₁ is selected from adenine methyl, uracil methyl, cytosine methyl, or guanine methyl modified by one or more of thiolation, halogenation, hydroxylation, amination, carboxylation, or methylation. Preferably, Ri is selected from adenine formyl, uracil formyl, cytosine formyl, or guanine formyl modified by one or more of thiolation, halogenation, hydroxylation, amination, carboxylation, or methylation.

R₂ is selected from H, C₁₂-C₅₀ alkyl, or C₁₂-C₅₀ alkyl modified by one or more of the following groups: hydroxyl, ether, carbonyl, amide, carboxyl, ester, amino, imino, tertiary amino, halogen, thio, thioether, sulfhydryl, sulphonic acid group, phospho, or phosphate groups.

Preferably, R₂ is selected from the following groups:

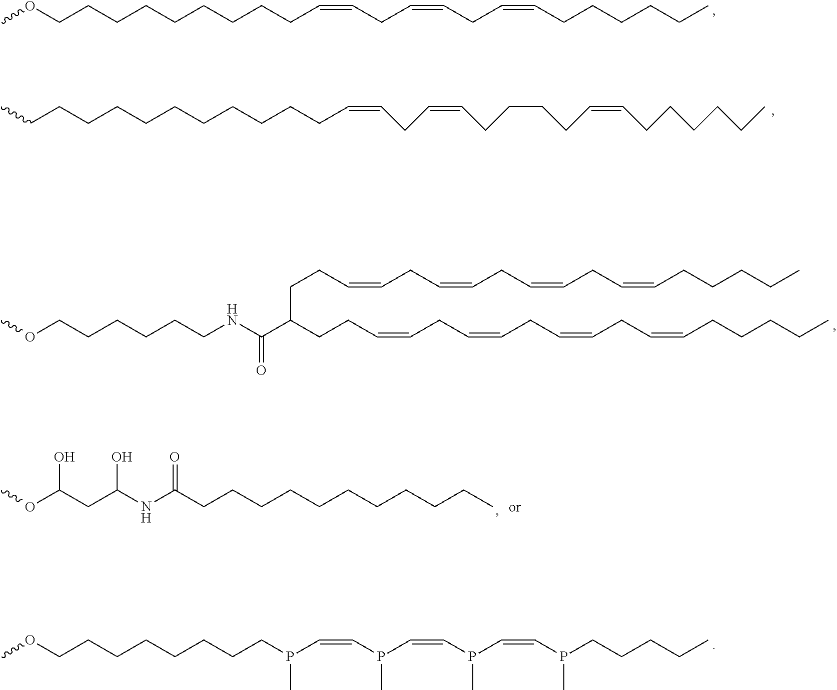

### EXAMPLES

### Example 1: Synthesis of Nucleosides

### Synthesis of Lipophilic Chain a

S1: Starting Material 1 (CAS No.: 55182-74-6) was converted to Intermediate 1 according to a method described in a reference (CN110028405A). Intermediate 1 was then used as a starting material to prepare Intermediate 2 according to the method reported in reference (Journal of Chemical Ecology, vol. 27, issue 4, (2001), p. 791-806).

S2: Starting Material 2 (CAS No.: 112-31-2) and Intermediate 2 were converted to Intermediate 3 according to the method described in a reference (European Journal of Organic Chemistry; vol. 9; (2000); p. 1821-1826).

S3: Intermediate 3 was further transformed into Lipophilic Chain a according to a method described in a reference (ChemCatChem; Vanka, Kumar, vol. 12, issue 14, (2020), p. 4586-4592), with a structure represented by Formula (III):

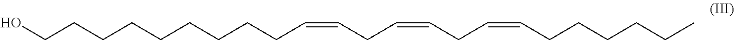 [SMILES: CCCCCC/C=C\C/C=C\C/C=C\CCCCCCCCCO]

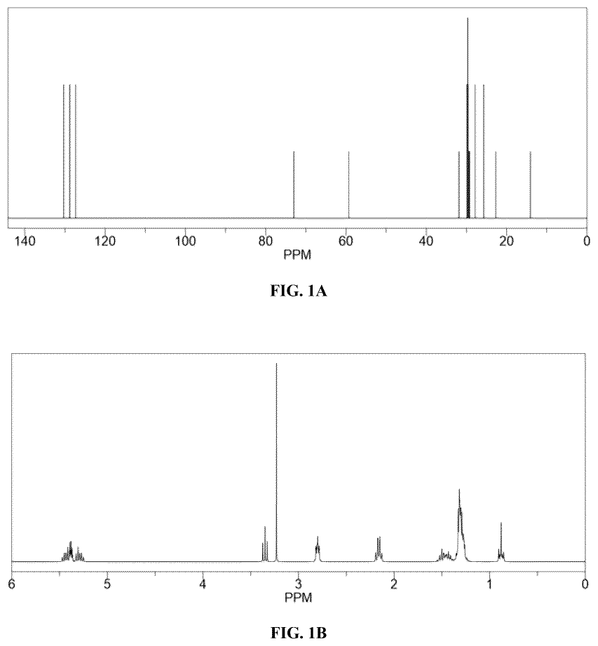

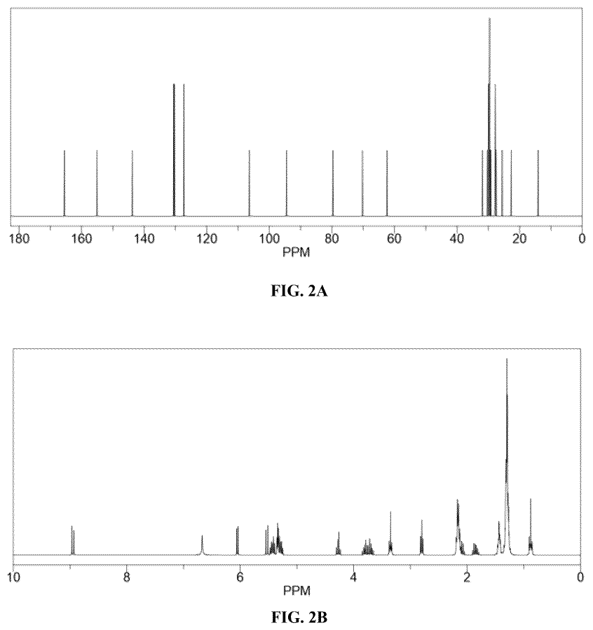

A ¹H-NMR spectrum of Lipophilic Chain a is shown in FIG. 1A, and a ¹³C-NMR spectrum of Lipophilic Chain a is shown in FIG. 1B.

### Synthesis of Lipophilic Chain b

S1: 3-buten-1-ol (2.77 mmol, 200 mg) was mixed with N,N-dimethylformamide (DMF, 2.0 mL, with a purity >99%) to obtain a mixture.

S2: 1-bromononane (355 μL, 1.85 mmol) and sodium hydride (NaH, 112 mg, 2.77 mmol) were added to the mixture obtained in S1 at room temperature, the mixture was stirred until the starting materials were completely consumed (monitored by thin-layer chromatography). After stirring, the reaction was quenched with water to obtain a mixture.

S3: The reaction mixture obtained in S2 was extracted with ethyl acetate/hexane (with a volume ratio of 10:1).

S4: An organic layer extracted in S3 was washed twice with water, and an organic layer was separated and extracted after each washing. The washed organic layer was dried over anhydrous sodium sulfate and filtered to obtain a filtrate, and the filtrate was concentrated under vacuum to obtain a crude product.

S5: The crude product was purified by silica gel column chromatography using hexane/ethyl acetate (with a volume ratio of 20:1) as eluent, to obtain a colorless oil (Intermediate 1).

S6: Intermediate 2 was prepared according to a method described in a reference (Liebigs Annalen der Chemie; 1; (1981); p. 92-98).

S7: Intermediate 1 and Intermediate 2 were reacted according to a method described in a reference (Journal of the American Chemical Society, vol. 123, issue 50, (2001), p. 12504-12509) to obtain Lipophilic Chain b, with a structure represented by Formula (IV).

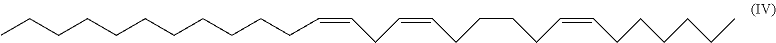 [SMILES: CCCCCC/C=C\CCCC/C=C\C/C=C\CCCCCCCCCCC]

A ¹H-NMR spectrum of Lipophilic Chain b is shown in FIG. 2A, and a ¹³C-NMR spectrum of Lipophilic Chain b is shown in FIG. 2B.

### Synthesis of Lipophilic Chain c

S1. Intermediate 1 was prepared according to the following references:
Chemistry and Industry (London, United Kingdom); (1959); p. 1288; Journal of the Chemical Society; (1961); p. 2779,2786;
Bioorganic and Medicinal Chemistry; vol. 15; 2; (2007); p. 854-867;
Journal of Organic Chemistry; vol. 63; 11; (1998); p. 3741-3744; and
Journal of Organic Chemistry; vol. 54; 23; (1989); p. 5522-5527.

S2: Intermediate 2 was prepared by mixing Compound (CAS No.: 2566-89-4) and Compound (CAS No.: 2134-29-4) for reaction according to a method described in the following references:
Angewandte Chemie-International Edition; vol. 54; 13; (2015); p. 4023-4027; and Angewandte Chemie; vol. 127; 13; (2015); p. 5.

S3: Intermediate 3 was prepared by mixing Intermediate 1 and Intermediate 2 for reaction according to a method described in a reference (WO2019010414A1).

S4. Intermediate 4 was prepared according to the following references:
Liebigs Annalen der Chemie; 11; (1992); p. 1113-1124; and
Monatshefte Fur Chemie; vol. 112; (1981); p. 825-840.

S5: Intermediate 3 and Intermediate 4 were reacted according to a method described in a reference (Journal of Medicinal Chemistry; vol. 40; 22; (1997); p. 3626-3634) to obtain Lipophilic Chain c, with a structure represented by Formula (V).

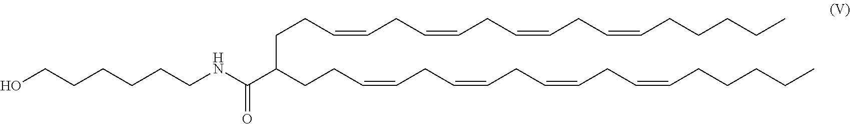 [SMILES: CCCCC/C=C\C/C=C\C/C=C\C/C=C\CCC(CC/C=C\C/C=C\C/C=C\C/C=C\CCCCC)C(=O)NCCCCCCO]

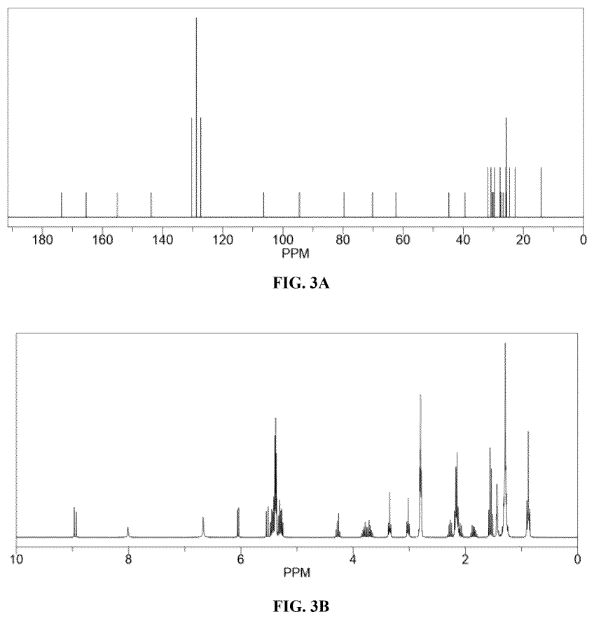

A ¹H-NMR spectrum of Lipophilic Chain c is shown in FIG. 3A, and a ¹³C-NMR spectrum of Lipophilic Chain c is shown in FIG. 3B.

### Synthesis of Lipophilic Chain d

S1: Starting Material 1 (CAS No.: 542-78-9) and Starting Material 2 (CAS No.: 1120-16-7) were mixed for reaction according to a method described in a reference (Tetrahedron Letters, vol. 48, issue 42, (2007), p. 7456-7459) to obtain Intermediate 1.

S2: Intermediate 1 was subjected to a reaction according to a method described in a reference document (Molecules; vol. 27; 9; (2022)) to obtain Lipophilic Chain d, with a structure represented by Formula (VI).

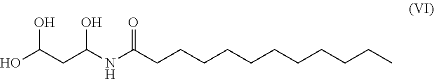 [SMILES: CCCCCCCCCCCC(=O)NC(O)CC(O)O]

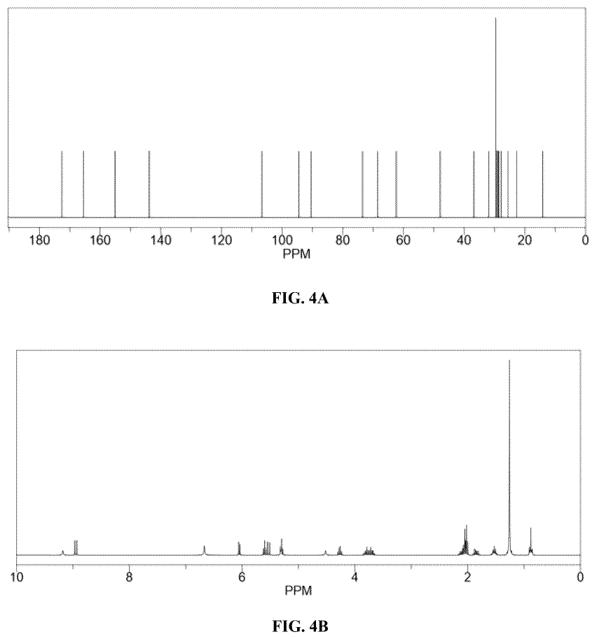

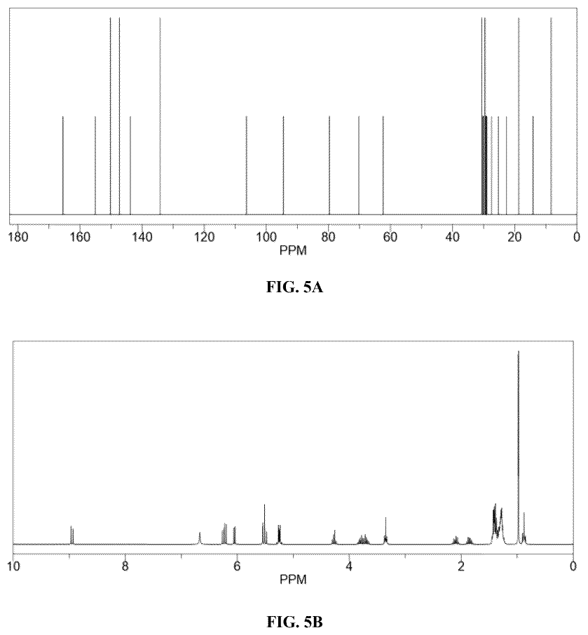

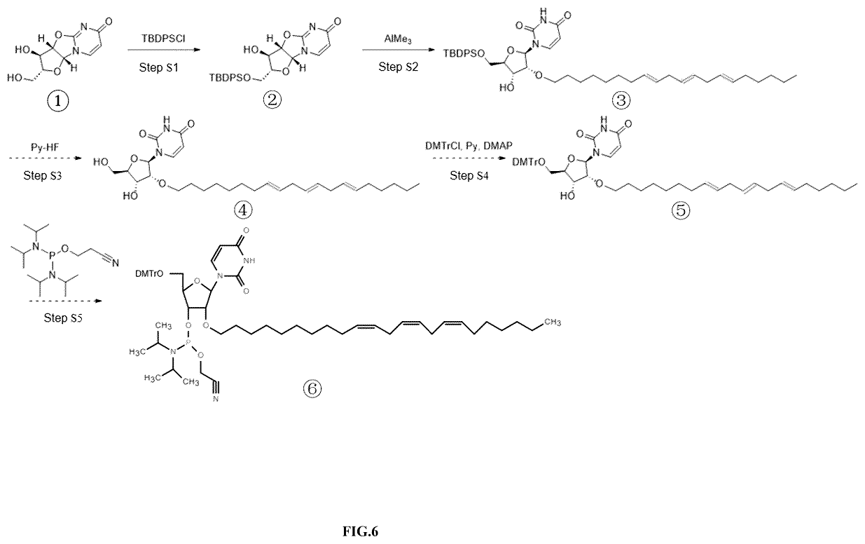

A ¹H-NMR spectrum of Lipophilic Chain d is shown in FIG. 4A, and a ¹³C-NMR spectrum of Lipophilic Chain d is shown in FIG. 4B.

### Synthesis of Lipophilic Chain e

Starting Material (CAS No.: 156-60-5) was subjected to a reaction according to a method described in a reference (Journal of Organic Chemistry; vol. 34; (1969); p. 1130-1133) to obtain Lipophilic Chain e, with a structure represented by Formula (VII).

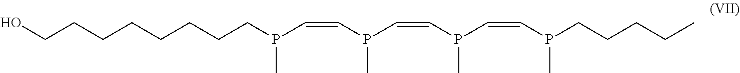 [SMILES: CCCCCP(C)/C=C\P(C)/C=C\P(C)/C=C\P(C)CCCCCCCCO]

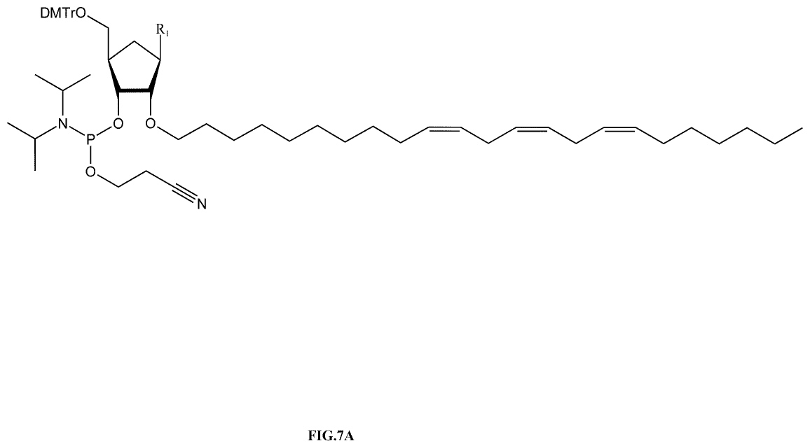

A ¹H-NMR spectrum of Lipophilic Chain e is shown in FIG. 5A, and a ¹³C-NMR spectrum of Lipophilic Chain e is shown in FIG. 5B.

### Synthesis of Nucleosides Containing Special Modifications

### Synthesis of Nucleoside A

Nucleoside A can be synthesized using Lipophilic Chain a and dehydrated nucleotide. The dehydrated nucleotide is selected from dehydrated adenosine, dehydrated uridine, dehydrated cytosine, dehydrated thymine, dehydrated guanine, or analogs thereof.

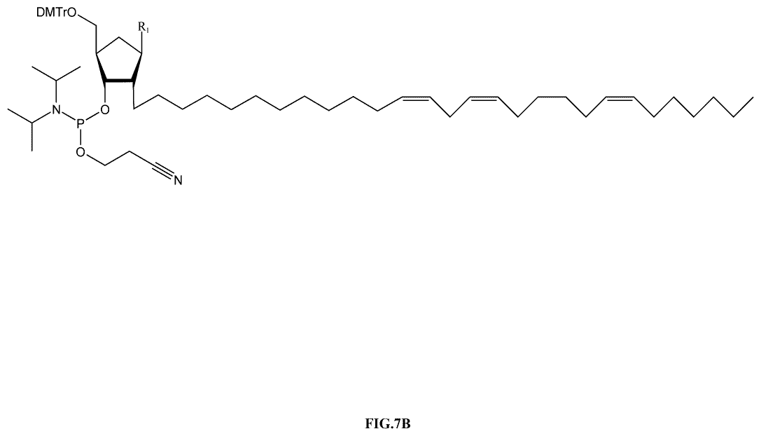

For example, Nucleoside A containing Lipophilic Chain a is synthesized using Lipophilic Chain a and dehydrated uridine. A synthesis process is shown in FIG. 6:

S1: Compound 1 (2,2′-dehydrated uridine, CAS No.: 3736-77-4) with a concentration of 0.06-0.1 M was added to 2 mL of tetrahydrofuran (THF), 100 μL of tert-Butylchlorodiphenylsilane (TBDPSCl, CAS No.: 58479-61-1) was added slowly and stirred at the same time mix thoroughly to obtain a reaction system. 100 μL of triethylamine was slowly added dropwise as a base catalyst to the reaction system to obtain a mixture, the mixture was continuously stirred to mix thoroughly, and incubated at room temperature overnight to generate a solution containing Compound 2.

S2: 10 μL of trimethylaluminum (CAS No. 75-24-1) with a concentration of 0.1 M was slowly added to the solution containing Compound 2 obtained in the step S1 and stirred to mix thoroughly to generate a solution containing Compound 3.

S3: 10 μL of pyridine hydrofluoride (CAS No. 62778-11-4) was slowly added to the solution containing Compound 3 obtained in the step S2 and stirred to mix thoroughly to generate a solution containing Compound 4.

S4: 100 μL of 4,4′-dimethoxytrityl chloride (DMTrCl, CAS No.: 40615-36-9) was slowly added to the solution containing Compound 4 obtained in the step S3, and a small amount of pyridine (CAS No. 110-86-1) and 4-dimethylaminopyridine (DMAP, CAS No.: 1122-58-3) were added as catalysts for reaction to generate a solution containing Compound 5.

S5: 50 μL of bis(diisopropylamino) (2-cyanoethoxy) phosphine (CAS No.: 102691-36-1) was added to the solution containing Compound 5 obtained in the step S4, and stirred to mix thoroughly to generate Nucleoside A.

### Synthesis of Nucleosides B-E

Nucleosides B-E were synthesized under conditions essentially same as those for nucleoside A, except that Lipophilic Chain a was replaced with one of Lipophilic Chains b-e.

TABLE 1
Some starting materials for synthesis of Nucleosides A-E

| Nucleosides | Some starting materials | | |
|---|---|---|---|
| Nucleoside A | Lipophilic Chain a | Ribose | Nitrogenous |
| Nucleoside B | Lipophilic Chain b | intermediate | base |
| Nucleoside C | Lipophilic Chain c | | |
| Nucleoside D | Lipophilic Chain d | | |
| Nucleoside E | Lipophilic Chain e | | |

Some starting materials for synthesis of Nucleosides A-E are summarized in Table 1.

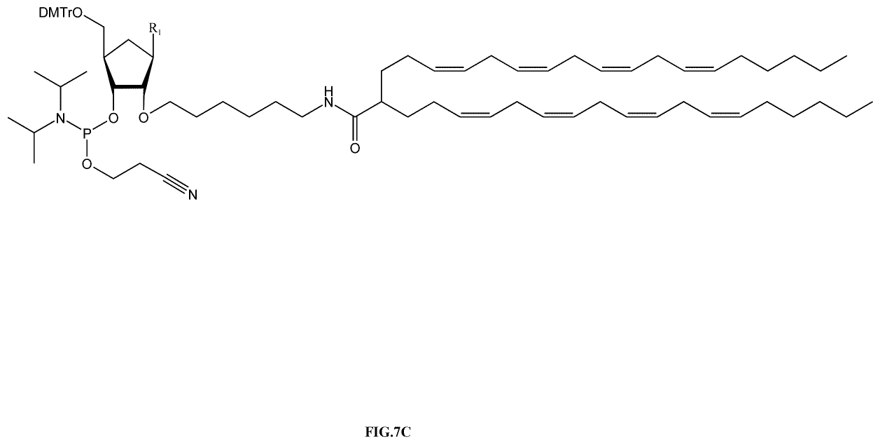

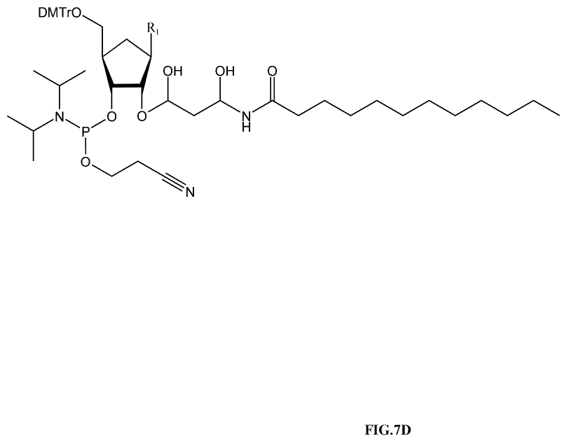

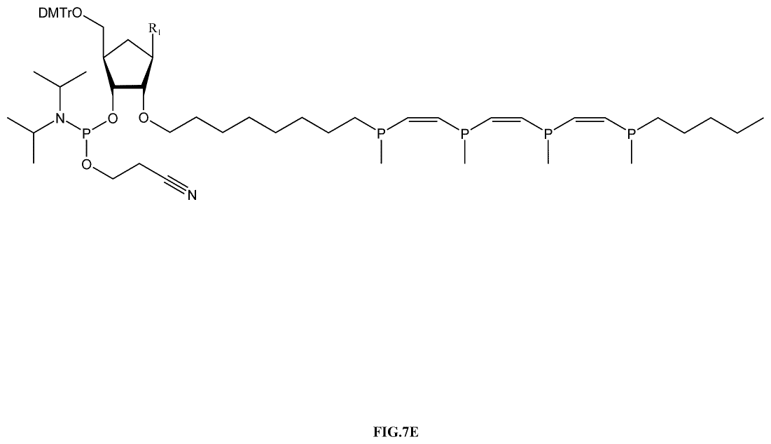

Nucleosides A-E with different specific modifications were synthesized using the above method, and their general structures are shown in FIG. 7.

### Example 2: Design and Synthesis of siRNA Sequences

Design of siRNA Sequences

siRNA-T1 (Target gene with a GenBank accession number: MH180383.1) has a sense strand with a sequence of 5′-GCCUGUAGCCCAUGUUGUATT-3′ (SEQ ID NO.1), and an antisense strand with a sequence of 5′-UACAACAUGGGCUACAGGCTT-3′ (SEQ ID NO.2). In the sense strand, a non-bridging oxygen atom of an α-phosphate group at bases 2, 3, 4, 6, 7, 9, 10, 11, 12, 13, 15, 16, 18, and 19 (counted from the 5′ end) was replaced with sulfur, i.e., phosphorothioate modification was applied. In addition, different types of lipophilic chain modifications were introduced for the siRNA-TI sequences. The sequence information is summarized in Table 2.

TABLE 2
[]siRNA-T1 modified sequence information

| S/N | Sense strand (5′-3′) | Antisense strand (5′-3′) |
|---|---|---|
| 1#(X) | GmC*C*U*GU*A*GC*C*C*A*U*GU*U*GU*A*TT | UmACAACAUGGGCUACAGGCTT |
| 2#(X) | GmC*C*U*GU*A*GC*C*C*A*U*GU*U*GU*AmTT | UmACAACAUGGGCUACAGGCmTT |
| 3#(X) | GmCm*C*U*GU*A*GC*C*C*A*U*GU*U*GU*A*TT | UmAmCAACAUGGGCUACAGGCTT |
| 4#(X) | GmCmC*U*GU*A*GC*C*C*A*U*GU*U*GUmAmTT | UmAmCAACAUGGGCUACAGGmCmTT |
| 5#(X) | GmC*C*U*GmU*A*GmC*C*C*A*U*GmU*U*GmU*A*TT | ACAACAUGGGCUACAGGCTT |
| 6#(X) | GmC*C*U*GmU*A*GmC*C*C*A*U*GmU*U*GmU*A*TT | UACAACAUGmGmGmCUACAGmGmCTT |

Notes:
“*” indicates that the nucleotide has a phosphorothioate modification;
“m” indicates that the nucleotide contains one of the special modifications A, B, C, D, or E;
“X” indicates that the type of special modification of the nucleotide in the RNA molecules corresponding to different Lipophilic Chains (X = A, B, C, D, or E), such as, 1#A;
When nucleotides are not specifically modified, RNA molecules are directly represented by 1#, 2#, 3#, 4#, 5#, or 6#.

Synthesis of siRNA Sequences

The siRNA sequence was synthesized by a phosphite triester method using five commercially available nucleosides AGCUT, together with Nucleosides A-E having different nitrogenous bases obtained in Example 1, where the synthesis was carried out by repeating four steps of “detritylation—coupling—oxidation—capping”, with one base added to the oligonucleotide chain upon completion of each cycle. Trichloroacetic acid/dichloromethane mixture was used as a deprotection reagent, 5-benzylthio-1H-tetrazole was used as an activator, iodine solution or (E)-N,N-dimethyl-N′-(3-thioxo-3H-1,2,4-dithiazol-5-yl) formamidine was used as an oxidation reagent, 2,2-dimethyl-N-(3-thioxo-3H-1,2,4-dithiazol-5-yl) propanamide (CAS No.: 56950-66-4) was used as a thiolation reagent, and acetic anhydride/tetrahydrofuran and N-methylimidazole was used as a capping reagent to perform the synthesis. The resulting synthesized products were then deprotected and annealed.

The annealed products were purified using high performance liquid chromatography (HPLC) to obtain different siRNA sequences.

Purification materials and parameters: A NanoQ-15L anion exchange column was used. Equilibration buffer: 100 mmol/L of tris (hydroxymethyl)aminomethane (pH 7.5), 10 mmol/L of ethylenediaminetetraacetic acid (pH 8.0) and 300 mmol/L of NaCl were mixed to obtain an equilibration buffer, with a pH of 9.0, and a conductivity of 32 mS/cm. Elution buffer: 100 mmol/L of tris (hydroxymethyl)aminomethane (pH 7.5), 10 mmol/L of ethylenediaminetetraacetic acid (pH 8.0) and 700 mmol/L of NaCl (pH 9.0) were mixed to obtain an elution buffer, with a conductivity of 65.2 mS/cm, and a flow rate of 10 mL/min.

### Example 3: Transfection of siRNA into Mouse Brain Cells

### Preparation of Mouse Brain Cells

Cerebral gray matter was collected from brains of B6 mice (3-4 months old, female, purchased from Shanghai Shengchang Biotechnology Co., Ltd.). Meninges, blood vessels, and fibrous components were carefully removed, and then washed once or twice with Hanks' solution. The washed gray matter tissues were suspended in 30-50 volumes of Hanks' solution and repeatedly pipetted to prepare a cell suspension. The suspension was transferred into a centrifuge tube and allowed to stand upright at room temperature for 5-10 minutes, during which cells or cell clumps settled naturally while other debris such as lipids floated. A supernatant was carefully removed, and this procedure was repeated 2-3 times. An appropriate volume of culture medium was added to the remaining sediment, which was filtered through a gauze or a mesh, cells after filtration was counted, and a cell density was adjusted. The mouse brain cells were seeded into a culture flask or dish and incubated in a 5% CO₂ incubator. An appropriate number of cells were seeded into a 12-well plate, ensuring that the cells reached approximately 50% confluence per well the following day.

Transfection of A-Modified siRNA

During transfection, one of six siRNA molecules (1 #A-6 #A) was added to the cells in each well. For each siRNA, high-dose and low-dose groups were established. The high-dose group was transfected at a concentration of 100 nM, and the low-dose group was transfected at a concentration of 30 nM. After 48 hours, RNA was extracted using TianGen RNA prep Pure Cell/Bacteria Kit (Cat. No. DP430) and reverse-transcribed to generate cDNA. (a sense strand of 1 #A is shown in SEQ ID NO.3, and an antisense strand of the same is shown in SEQ ID NO.4; a sense strand of 2 #A is shown in SEQ ID NO.5, and an antisense strand of the same is shown in SEQ ID NO.6; a sense strand of 3 #A is shown in SEQ ID NO.7, and an antisense strand of the same is shown in SEQ ID NO.8; a sense strand of 4 #A is shown in SEQ ID NO.9, and an antisense strand of the same is shown in SEQ ID NO.10; a sense strand of 5 #A is shown in SEQ ID NO.11, and an antisense strand of the same is shown in SEQ ID NO.12; and a sense strand of 6 #A is shown in SEQ ID NO.11, and an antisense strand of the same is shown in SEQ ID NO.13)

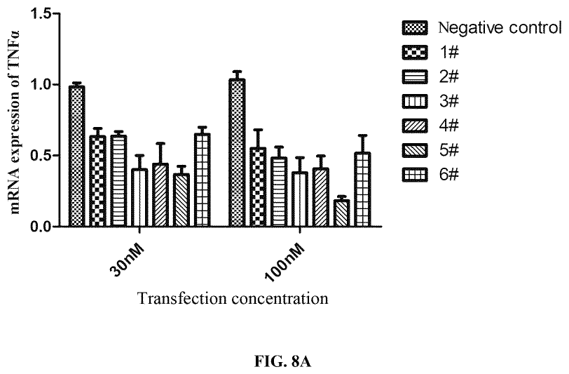

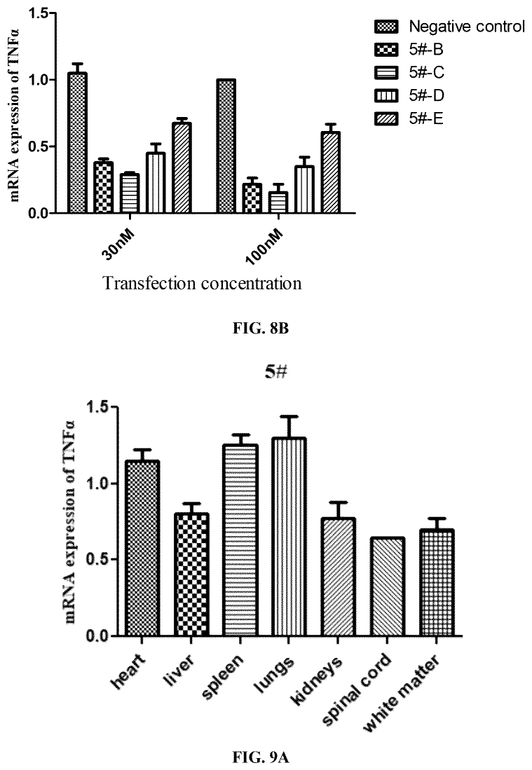

TNF-α mRNA expression was assessed by quantitative PCR (Q-PCR), with the results shown in FIG. 8A. All six siRNA molecules effectively knocked down TNF-α mRNA expression at transfection concentrations of 30 nM and 100 nM. At both low and high transfection concentrations, 5 #A siRNA molecule exhibited the most potent knockdown effect, reducing the target mRNA expression to approximately 36% and 18.3% of the control level, respectively. Since 5 #A siRNA molecule exhibited the best specific knockdown of TNF-α mRNA, the 5 #A siRNA molecule was selected for subsequent validation.

Transfection of B-Modified to E-Modified siRNA

Based on the results of transfection of A-modified siRNA, four siRNA, that is, 5 #B-5 #E siRNAs, were used for transfection experiments.

During transfection, one of 5 #B, 5 #C, 5 #D, or 5 #E siRNAs was added to the cells in each well. For each siRNA, high-dose and low-dose groups were established. The high-dose group was transfected at a concentration of 100 nM, and the low-dose group was transfected at a concentration of 30 nM. After 48 hours, RNA was extracted using TianGen RNA prep Pure Cell/Bacteria Kit (Cat. No. DP430) and reverse-transcribed to generate cDNA. (a sense strand of 5 #B is shown in SEQ ID NO.14, and an antisense strand of the is shown in SEQ ID NO.12; a sense strand of 5 #C is shown in SEQ ID NO.15, and an antisense strand of the same is shown in SEQ ID NO.12; a sense strand of 5 #D is shown in SEQ ID NO. 16, and an antisense strand of the same is shown in SEQ ID NO.12; and a sense strand of 5 #E is shown in SEQ ID NO.17, and an antisense strand of the same is shown in SEQ ID NO.12.)

TNF-α mRNA expression was assessed by quantitative PCR (Q-PCR), with the results shown in FIG. 8B. All four siRNA molecules effectively knocked down TNF-α mRNA expression at transfection concentrations of 30 nM and 100 nM. At both low and high transfection concentrations, 5 #C siRNA exhibited the most potent knockdown effect, reducing the target mRNA expression to approximately 29% and 15.5% of the control level, respectively. The knockdown efficiency decreased in the following order: 5 #C siRNA>5 #B siRNA>5 #D siRNA>5 #E siRNA.

### Example 4: Effect of Intrathecal Injection of siRNA Molecules on Target Gene mRNA Expression in Mice

Six siRNA molecules, that is, 5 #(unmodified), 5 #A, 5 #B, 5 #C, 5 #D, and 5 #E, were each formulated into sterile preparations using sterile water for injection as a solvent. To formulate the preparation, all components were added to a portion of the solvent based on the final volume, including 2 mg/mL siRNA, 0.2 mg/mL calcium chloride dihydrate, 0.1 mg/mL sodium dihydrogen phosphate, 0.16 mg/mL magnesium chloride hexahydrate, 0.03 mg/mL sodium phosphate, and 0.22 mg/mL potassium chloride. After complete dissolution, the solution was diluted with a solvent to near the final volume, and a pH was adjusted to 6.9-7.1 using 1 mg/mL HCl or NaOH. B6 mice were intrathecally injected with 50 μL of the siRNA preparation. 24 hours after the injection, the mice were euthanized. Heart, liver, spleen, lung, kidney, spinal cord, and cerebral white matter of each mouse were removed. In addition, organs from blank mice were also removed and taken as controls, and immediately frozen in liquid nitrogen.

Tweezers and scissors were sterilized using 75% ethanol to minimize RNase contamination. A 2 mL centrifuge tube was placed on ice, and an appropriate volume of PBS buffer was added into the centrifuge tube to prepare a tissue homogenate. A mass-to-volume ratio of the tissue homogenate required for the experiment was approximately 10%. Skin samples to be ground were taken out from a −80° C. freezer, and placed in a centrifuge tube on ice. The skin samples were then cut into small pieces using sterilized tweezers and scissors. Sterilized zirconia grinding ball was placed into centrifuge tubes, with one ball per each tube. The tubes were placed in a grinder for grinding at a frequency of 1000 vibrations/minute for 5 minutes at −20° C. After grinding, the tubes were placed in a pre-cooled high-speed centrifuge and centrifuged at 4000 rpm for 30 minutes. A resulting supernatant was removed with a pipette and transferred into a new centrifuge tube. Total RNA was extracted using TianGen RNA prep Pure Cell/Bacteria Kit (Cat. No. DP430), and TNF-α mRNA expression in each organ was assessed by the Q-PCR.

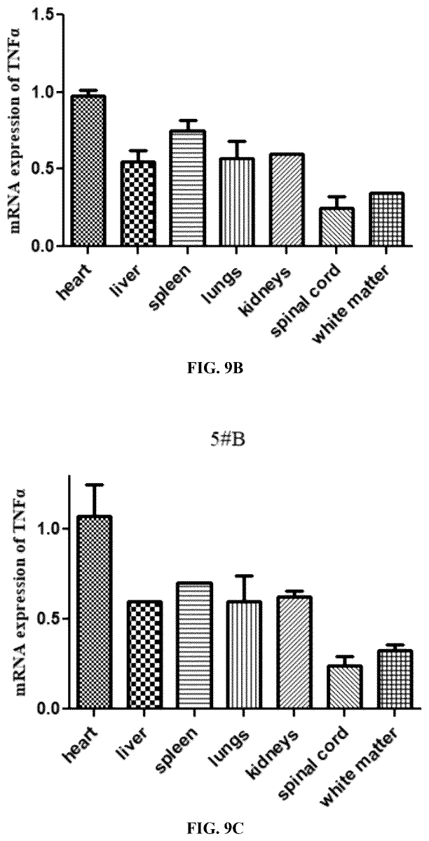

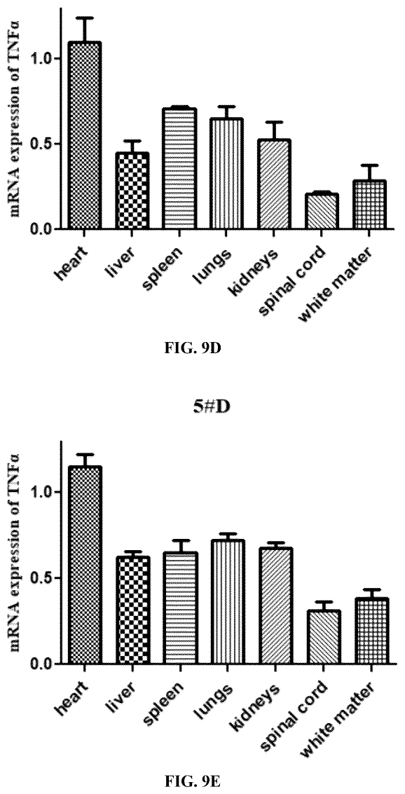

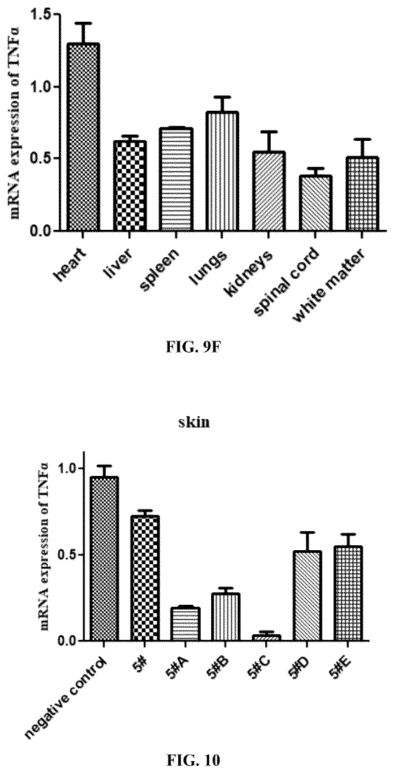

After intrathecal injection of 5 #siRNA, the effect on target gene mRNA expression in mice is shown in FIGS. 9A-F. The results indicated that 5 #siRNA had almost no knockdown effect in the heart, spleen, and lung, and moderate knockdown in the liver, kidney, spinal cord, and cerebral white matter, and the knockdown rates in all organs were lower than 50%. In contrast, 5 #A-5 #E siRNAs showed minimal knockdown in the heart, but produced varying degrees of knockdown effect in the liver, spleen, lungs, kidney, spinal cord, and cerebral white matter, generally outperforming 5 #siRNA. Among them, 5 #C siRNA exhibited the most knockdown effect, with knockdown effect in the liver of about 45%, in the spleen of about 71%, in the lung of about 65%, in the kidney of about 52.5%, in the spinal cord of about 21%, and in the cerebral white matter of about 28.5% (FIG. 9D).

In summary, the siRNA molecules with special modifications of the present disclosure, when intrathecally administered, can effectively reduce the target gene mRNA expression in the liver, spleen, lungs, spinal cord, and cerebral white matter. Compared with the unmodified siRNA, these modified molecules demonstrate significantly enhanced efficacy, and excellent lipophilicity.

### Example 5: Effect of Intradermal Injection of siRNA Molecules on Target Gene mRNA Expression in Mice

Six siRNA molecules 5 #A, 5 #B, 5 #C, 5 #D, 5 #E, and 5 #(unmodified) were each formulated into sterile preparations using the same method as described in Example 4. B6 mice were intradermally injected with 50 μg of the siRNA preparation. 24 hours after the injection, the mice were euthanized. Skin at the injection site of each mouse was removed. In addition, skin (including both epidermis and dermis) from blank mice was removed and taken as a control, and immediately frozen in liquid nitrogen.

Tweezers and scissors were sterilized using 75% ethanol to minimize RNase contamination. A 2 mL centrifuge tube was placed on ice, and an appropriate volume of PBS buffer was added into the centrifuge tube to prepare a tissue homogenate. A mass-to-volume ratio of the tissue homogenate required for the experiment was approximately 10%. Skin samples to be ground were taken out from a −80° C. freezer, and placed in a centrifuge tube on ice. The skin samples were then cut into small pieces using sterilized tweezers and scissors. Sterilized zirconia grinding ball was placed into centrifuge tubes, with one ball per each tube. The tubes were placed in a grinder for grinding at a frequency of 1000 vibrations/minute for 5 minutes at −20° C. After grinding, the tubes were placed in a pre-cooled high-speed centrifuge and centrifuged at 4000 rpm for 30 minutes. A resulting supernatant was removed with a pipette and transferred into a new centrifuge tube. Total RNA was extracted using TianGen RNA prep Pure Cell/Bacteria Kit (Cat. No. DP430), and TNF-α mRNA expression in each organ was assessed by the Q-PCR.

After intradermal injection of siRNA, the effect on target gene mRNA expression in mouse skin is shown in FIG. 10. The results indicated that 5 #reduced mRNA expression in the skin tissue to approximately 67.5%. All five specially modified siRNA molecules effectively reduced mRNA expression, with effect outperforming 5 #siRNA. The remaining mRNA expression levels after knockdown for five siRNA molecules 5 #A˜5 #E were approximately 19.5%, 27.5%, 3.5%, 52.5%, and 55.0%, respectively, with 5 #C siRNA exhibiting the highest knockdown effect. The knockdown effect ranked from high to low was: 5 #C siRNA>5 #A siRNA>5 #B siRNA>5 #D siRNA>5 #E siRNA.

The experiment results indicated that the siRNA molecules with special modifications of the present disclosure, when administered intradermally, can effectively reduce the target gene mRNA expression in the skin tissue. Compared with the unmodified siRNA, these modified molecules demonstrate significantly enhanced efficacy, excellent lipophilicity and transdermal permeability.

### Example 6: Cytotoxicity of siRNA Molecules

HaCat cells were transfected with five siRNA molecules 5 #A, 5 #B, 5 #C, 5 #D, and 5 #E molecules, respectively, and cytotoxicity was then assessed using an assay kit (Cat. No.: C0038, purchased from Beyotime Biotechnology), with specific operating steps as follows:

S1: On Day 1, an appropriate number of cells were seeded into a 96-well plate, ensuring that the cells reached approximately 50% confluence per well the following day.

S2: On Day 2, when the cells reached approximately 50%, each well was transfected with one of the five siRNA molecules 5 #A-5 #E preparations (the preparation method was the same as that described in Example 4) in each well, with a transfection concentration of 100 pmol siRNA.

S3: The transfected cells were incubated overnight at 37° C. in a 5% CO₂ environment.

S4: After 2 hours, 4 hours, 6 hours, 8 hours, 16 hours and 24 hours, the absorbance of each well was measured at 450 nm using a microplate reader.

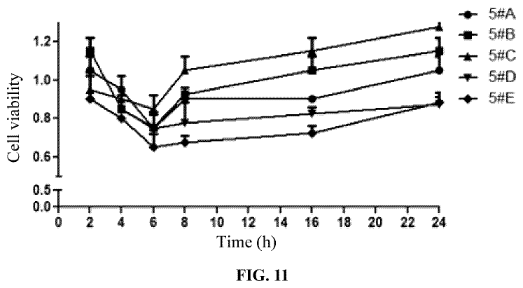

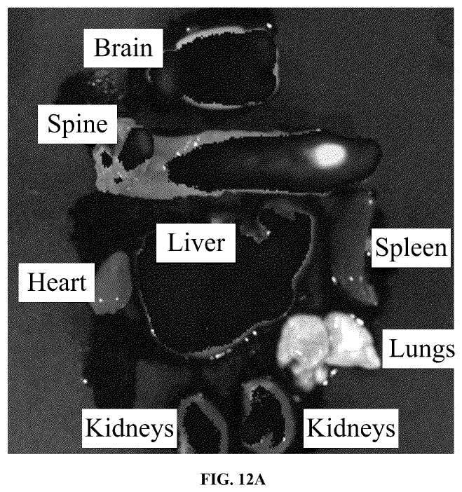

As shown in FIG. 11, the five siRNA molecules exhibited slight cytotoxicity 4-8 hours after transfection, followed by gradual recovery of cell viability. The results indicated that all five siRNA molecules exhibited minimal cytotoxicity.

### Example 7: Evaluation of siRNA Molecules Delivery and In Vivo Distribution

The sense strand of 5 #A siRNA, labeled with carboxyfluorescein (FAM) at the 3′-end, was synthesized using a controlled pore glass (CPG) column, and then formulated into a sterile preparation (the preparation method was the same as that described in Example 4). B6 mice were intrathecally injected with 50 μL of the siRNA preparation. 3 hours after the injection, the mice were euthanized. Heart, liver, spleen, lung, kidney, spinal cord, and cerebral white matter of each mouse were removed for tissue fluorescence imaging.

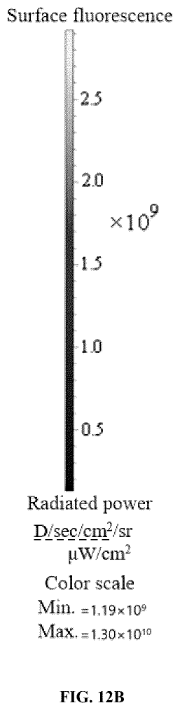

As shown in FIGS. 12A-B, an intensity of fluorescence signals, from strongest to weakest, was observed in the spinal cord, brain, liver, and kidney, while no significant signals were detected in the heart, spleen, or lung. The results indicated that 5 #A siRNA, when administered intrathecally, rapidly distributes in spinal cord tissue and exhibits excellent distribution properties.

In summary, the present disclosure increases the lipophilicity of siRNA sequences through the modification of lipophilic chains. The modified siRNA molecules achieve excellent carrier-free delivery efficiency and in vivo distribution via intrathecal injection, intradermal injection, or other administration routes. The molecules effectively knock down target gene expression while maintaining good tolerability and safety.

The above embodiments are merely preferred embodiments to fully illustrate the technical solutions and effects of the present disclosure. The purpose of the embodiments is to enable those skilled in the art to understand and implement the present disclosure, and should not be construed as limiting the scope of protection of the present disclosure. Equivalents, substitutions, or variations made by those skilled in the art based on the present disclosure are intended to fall within the scope of protection of the present disclosure.

What is claimed is:

1. A nucleoside, wherein the nucleoside has a structure as represented by Formula (I), and comprises R₁, R₃, and lipophilic R₂, wherein: 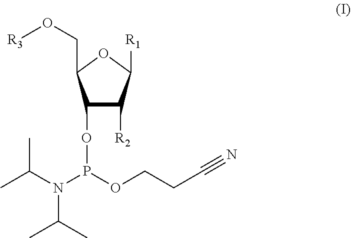
R₁ is selected from adenine, uracil, cytosine, or guanine,
adenine, uracil, cytosine, or guanine modified by one or more of thiolation, halogenation, hydroxylation, amination, carboxylation, or methylation,
adenine methyl, uracil methyl, cytosine methyl, or guanine methyl modified by one or more of thiolation, halogenation, hydroxylation, amination, carboxylation, or methylation, or
adenine formyl, uracil formyl, cytosine formyl, or guanine formyl modified by one or more of thiolation, halogenation, hydroxylation, amination, carboxylation, or methylation;
R₂ is selected from the following groups: 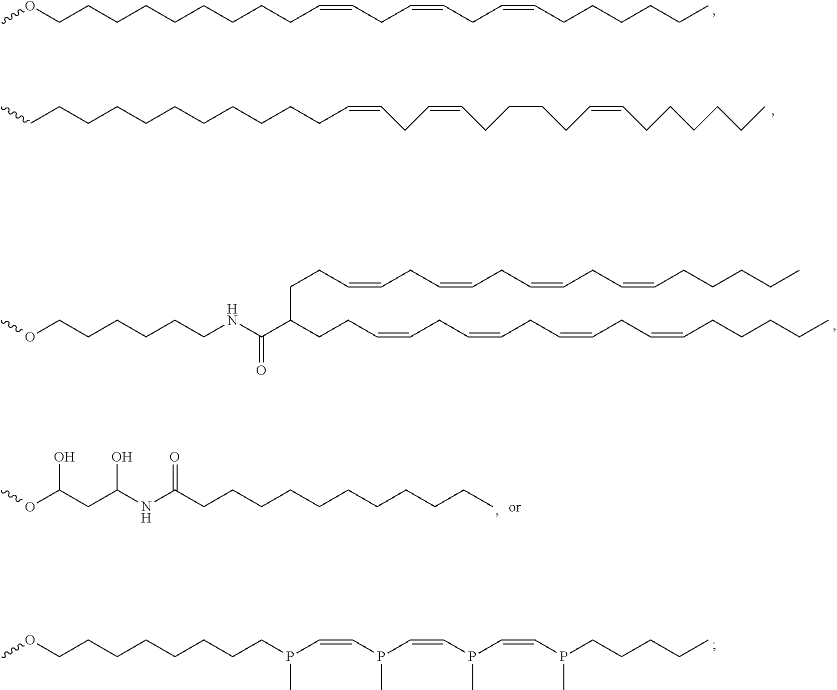
R₃ is selected from 4,4′-dimethoxytrityl, 4-monomethoxytrityl, 4,4′,4″-trimethoxytrityl, pivaloyloxymethyl, tert-butyldimethylsilyl, 9-fluorenylmethoxycarbonyl, phenoxyacetyl, 4-tert-butylphenoxyacetyl, 2-cyanoethoxy-N,N-diisopropylaminophosphino, 3-pentanedione, acetyl, benzoyl, 2-cyanoethyl, N,N-dibutylaminocarbonyl, N,N-dimethylaminocarbonyl, isobutyryl, 2-(2-nitrophenyl)-propoxycarbonyl, triethylammonium, trifluoroacetyl, triisopropylsiloxymethyl, para-isopropylphenoxyacetyl, O-acetal levulinyl ester, phenylacetyl, or 1,1′,3,3′-tetra-isopropyl disiloxanyl.

2. A double-stranded siRNA, comprising a sense strand and an antisense strand, wherein a sequence of the sense strand is shown in SEQ ID NO. 1, from the 5′ end to the 3′ end is 5′-GCCUGUAGCCCAUGUUGUATT-3′, and a sequence of the antisense strand is shown in SEQ ID NO. 2, from the 5′ end to the 3′ end is 5′-UACAACAUGGGCUACAGGCTT-3′; and a nucleotide in the double-stranded siRNA is specially modified, and the specially modified nucleotide comprises R₁ and lipophilic R₂, having a structure as represented by Formula (II): 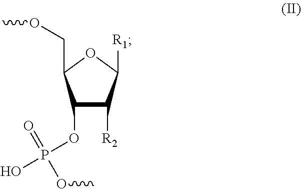
R₁ is selected from adenine, uracil, cytosine, or guanine,
adenine, uracil, cytosine, or guanine modified by one or more of thiolation, halogenation, hydroxylation, amination, carboxylation, or methylation,
adenine methyl, uracil methyl, cytosine methyl, or guanine methyl modified by one or more of thiolation, halogenation, hydroxylation, amination, carboxylation, or methylation, or
adenine formyl, uracil formyl, cytosine formyl, or guanine formyl modified by one or more of thiolation, halogenation, hydroxylation, amination, carboxylation, or methylation;
R₂ is selected from the following groups:

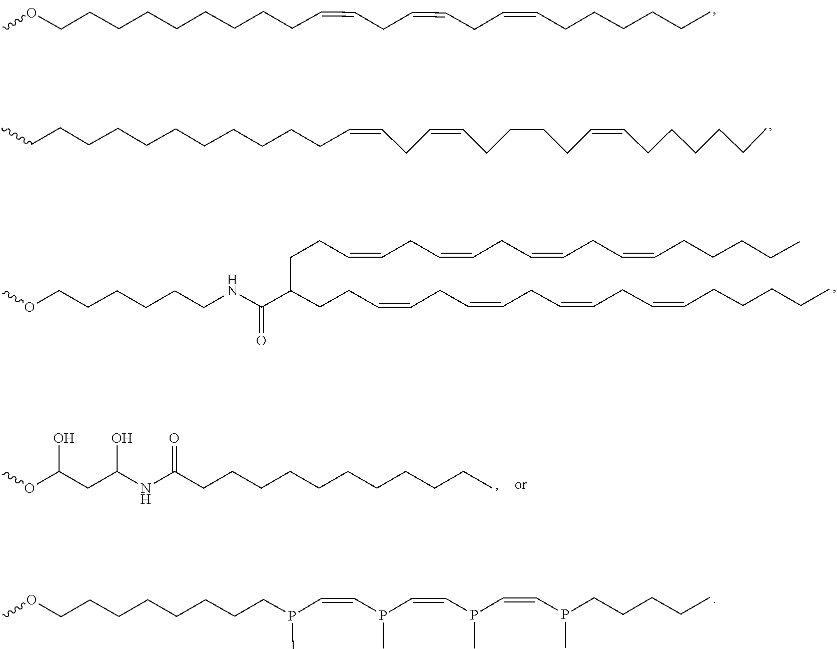

3. The double-stranded siRNA according to claim 2, wherein the specially modified nucleotide is located at one or more positions selected from positions 1, 2, 5, 8, 14, 17, 18, or 19 in a 5′ to 3′ direction of the sense strand whose nucleotide sequence is shown in SEQ ID NO.1.

4. The double-stranded siRNA according to claim 2, wherein the specially modified nucleotide is located at one or more positions selected from positions 1, 2, 9, 10, 11, 17, 18, or 19 in a 5′ to 3′ direction of the antisense strand whose nucleotide sequence is shown in SEQ ID NO.2.

5. The double-stranded siRNA according to claim 3, wherein the specially modified nucleotide is located at:
a position 1 in the 5′ to 3′ direction of the sense strand whose nucleotide sequence is shown in SEQ ID NO.1;
positions 1 and 19 in the 5′ to 3′ direction of the sense strand whose nucleotide sequence is shown in SEQ ID NO.1;
positions 1 and 2 in the 5′ to 3′ direction of the sense strand whose nucleotide sequence is shown in SEQ ID NO.1;
positions 1, 2, 18 and 19 in the 5′ to 3′ direction of the sense strand whose nucleotide sequence is shown in SEQ ID NO.1; or
positions 1, 5, 8, 14 and 17 in the 5′ to 3′ direction of the sense strand whose nucleotide sequence is shown in SEQ ID NO.1.

6. The double-stranded siRNA according to claim 4, wherein the specially modified nucleotide is located at:
a position 1 in the 5′ to 3′ direction of the antisense strand whose nucleotide sequence is shown in SEQ ID NO.2;
positions 1 and 19 in the 5′ to 3′ direction of the antisense strand whose nucleotide sequence is shown in SEQ ID NO.2;
positions 1 and 2 in the 5′ to 3′ direction of the antisense strand whose nucleotide sequence is shown in SEQ ID NO.2;
positions 1, 2, 18 and 19 in the 5′ to 3′ direction of the antisense strand whose nucleotide sequence is shown in SEQ ID NO.2; or
positions 9, 10, 11, 17 and 18 in the 5′ to 3′ direction of the antisense strand whose nucleotide sequence is shown in SEQ ID NO.2.

7. The double-stranded siRNA according to claim 2, wherein the specially modified nucleotide is located at:
a position 1 in the 5′ to 3′ direction of the sense strand whose nucleotide sequence is shown in SEQ ID NO.1, and position 1 in the 5′ to 3′ direction of the antisense strand whose nucleotide sequence is shown in SEQ ID NO.2;
positions 1 and 19 in the 5′ to 3′ direction of the sense strand whose nucleotide sequence is shown in SEQ ID NO.1, and positions 1 and 19 in the 5′ to 3′ direction of the antisense strand whose nucleotide sequence is shown in SEQ ID NO.2;
positions 1 and 2 in the 5′ to 3′ direction of the sense strand whose nucleotide sequence is shown in SEQ ID NO.1, and positions 1 and 2 in the 5′ to 3′ direction of the antisense strand whose nucleotide sequence is shown in SEQ ID NO.2;
positions 1, 2, 18 and 19 in the 5′ to 3′ direction of the sense strand whose nucleotide sequence is shown in SEQ ID NO.1, and positions 1, 2, 18 and 19 in the 5′ to 3′ direction of the antisense strand whose nucleotide sequence is shown in SEQ ID NO.2;
positions 1, 5, 8, 14 and 17 in the 5′ to 3′ direction of the sense strand whose nucleotide sequence is shown in SEQ ID NO.1; or
positions 1, 5, 8, 14 and 17 in the 5′ to 3′ direction of the sense strand whose nucleotide sequence is shown in SEQ ID NO.1, and positions 9, 10, 11 17 and 18 in the 5′ to 3′ direction of the antisense strand whose nucleotide sequence is shown in SEQ ID NO.2.

8. The double-stranded siRNA according to claim 2, wherein a non-bridging oxygen atom of a phosphate group attached to a 3′-carbon atom in nucleoside monophosphate at positions 2, 3, 4, 6, 7, 9, 10, 11, 12, 13, 15, 16, 18 and 19 of the sense strand whose nucleotide sequence is shown in SEQ ID NO.1 is substituted with a sulfur atom.

9. A pharmaceutical composition, comprising a therapeutically effective amount of the double-stranded siRNA according to claim 2, as well as a pharmaceutically acceptable carrier, a solvent or an excipient.
